(19) United States
(12) Patent Application Publication
(10) Pub. No.: US 2023/0248914 A1
(43) Pub. Date: Aug. 10, 2023

(54) Reusable Drug Administration Device using Replaceable Cannula Technology

(71) Applicant: Rashmika Maria Reddy-Mazzitello, Frisco, TX (US)

(72) Inventor: Rashmika Maria Reddy-Mazzitello, Frisco, TX (US)

(21) Appl. No.: 18/191,838

(22) Filed: Mar. 28, 2023

(51) Int. Cl. A61M 5/24; A61M 5/172

(52) U.S. Cl. CPC A61M 5/24; A61M 5/172; A61M 2205/3584

(57) ABSTRACT

A drug-administering, transdermal pod that uses Bluetooth motorized sensor technology to mechanize a replaceable subcutaneous cannula and refillable drug cartridge through user input via a mobile app.

### BACKGROUND OF THE INVENTION

The present device is in the field of drug administrative devices and specifically pertains to a bluetooth commanded device that injects a replaceable subcutaneous cannula, along with a refillable drug cartridge and sterile cannula replacement pen.

In the art of drug administration devices lies a current technology that consists commonly with similar structures of variant cannulas, whether intravenous, subcutaneous or another type, and drug cartridges. Such technologies require wireless devices or pods to be replaced once or more within periods of 2 to 10 days depending on the device itself, the medication administered, rate of prescription, and other factors due to health risks pertaining to infection by long term injection usage and impaired absorption at the injection site from prolonged subcutaneous use.

One problem with the replacement periods of existing devices regards the expenses of replacing the entire device technology every 2 to 10 days. With wireless drug administering pods costing consumer prices of at least $25 and beyond $800, the costs of disposable devices easily amounts to over $1000 annually at any price. Prices for the device do not include patient costs for the prescription drug.

The extensive patient costs of the device contribute significantly to the epidemic of consumers who lack safe treatment to conditions that would otherwise be treatable with orderly maintenance and treatment from the device. The market prices for existing devices prove unreasonable to many lower class and underprivileged consumers who cannot afford to constantly replace the technology, and effects of this can be seen on communities who are imposed with health threats from incorrect usage of the device or no treatment. In many instances, underprivileged individuals utilize the devices for a prolonged period, being longer than 10 days, due to troubles in affording replacements. These actions and those similar lead to severe infections and further health crises from keeping the cannula under the skin for too long.

Another problem exists in the material usage being exerted by the constant device replacements. Current replacement technology establishes large amounts of medical waste from each 2-10 day replacement, and are developed and designed in unnecessary and inconvenient ways in regards to the environment.

Therefore, what is clearly needed is a drug administrative device that solves the problems mentioned above.

### SUMMARY OF THE INVENTION

In response to the persisting problems, technological implementations including a series of cannula replacement mechanisms, refillable drug cartridge, and sterile cannula injector have been utilized within the device. By providing a cannula replacement mechanism consisting of left and right motor circuits that work to push a cannula upwards and out of the device as well as contracting the motors to push the cannula into the skin, the device reveals a single use replaceable cannula that significantly reduces medical waste and patient expenses by providing an alternative to the device replacement requirements. By requiring the sole replacement of the cannula in contrast to the replacement of the entire device technology, the device limits patient expenses to the replacement factors of a sole cannula replacement every 2-10 days, thus costing significantly less than the existing alternative of the entire device. The same innovation reduces the rapid waste of a working device by allowing the patient to continue using their device throughout the periodic cannula replacements instead of disposing of the device frequently. In addition, a refillable drug cartridge in cooperation with an injection and cleaning port utilizing a push-door mechanism exists to synchronize with the device reuse and provide a way to refill the device of the prescription drug when needed and maintain sanitary device condition through easy access for cleaning and sanitization when necessary. The sterile cannula injector synergizes with the cannula replacement mechanisms to offer a sterile and safe way for administering the new cannula replacement. Consisting of a pen-like outer casing and an intrinsic sterilized polypropylene hub, the cannula is stored within the structure and is injected using a pen-like insertion mechanism into the corresponding push door cannula insertion port. The cannula, discarding the intrinsic sheath, is then inserted and pushed into the grip of the left and right motors for skin insertion when commanded.

The previous innovations are utilized within the device to provide a more convenient, affordable, eco friendly, and user-friendly experience to patients and consumers.

### BRIEF DESCRIPTION OF DRAWINGS

These and other features and advantages of the various embodiments disclosed herein will be better understood with respect to the following description and drawings, in which like numbers refer to like parts throughout, and in which:

FIG. 1 depicts an exploded view and internal representation of the multiple inner schemes of the device such being the cartridge, insertion, battery, sensor, motor, adhesion, and cannula schemes.

FIG. 2 depicts an exploded and top-outer view of the device with illustration of the appearance of the device’s adhesives, drug cartridge injection and refill point and cannula replacement port.

### DETAILED DESCRIPTION OF THE INVENTION

The exemplary embodiments of the present invention deliver prescription drugs to the subcutaneous layer of the skin via a 38 mm replaceable cannula. By utilizing a sterile cannula injector and a pH skin color configuring adhesive, safe and proper insertion and maintenance of the inserted replaceable cannula in the subcutaneous space is ensured, while maintaining a degree of comfort to the user.

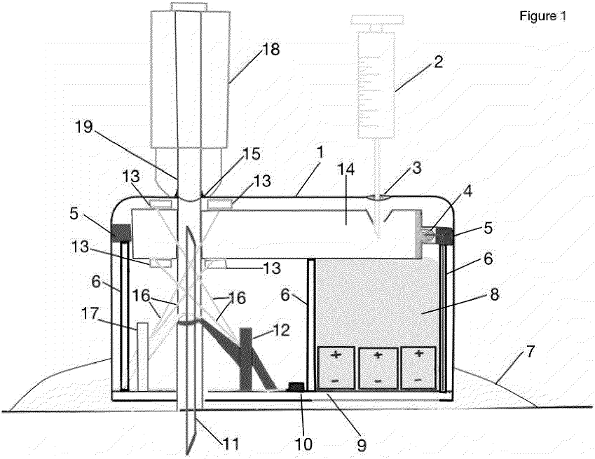

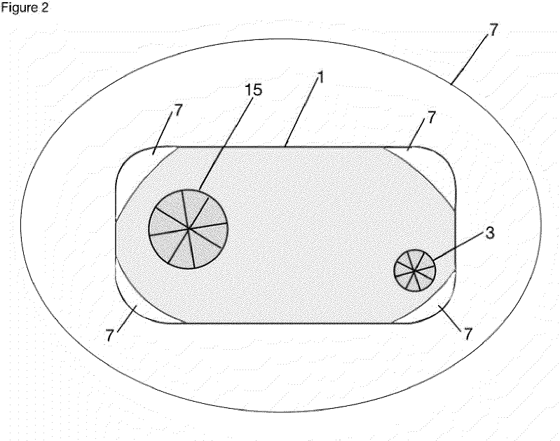

While FIG. 2 depicts the external aspects of the device, such being a pH skin color configuring adhesive 7, a device encasing body 1, a cannula replacement port 15, and drug cartridge injection and refill point 3, FIG. 1 depicts the internal mechanisms of the device encasing body 1, including but not limited to an exemplary infusion set mechanism consisting of a 38 mm replaceable cannula 11, cannula replacement port 15, left motor mechanism 17, right motor mechanism 12, cartridge expander gates 13, and set of cartridge gate expanding crossbars 16. The cartridge gate expanding crossbars 16 and cartridge expander gates 13 may also exist within the refillable drug cartridge mechanism consisting of a 10.1 mL/1010 IU drug cartridge 14, drug injection syringe 2, cartridge support bevels 5, drug cartridge injection and refill point 3, and micro ultrasonic level drug cartridge content level sensing transmitter 4. Synergizing with the infusion set mechanism is the cannula injector mechanism, similarly consisting of but not limiting to a 38 mm replaceable cannula 11 and drug cartridge injection and refill point 3, in addition to a sterile cannula injector 18 and an intrinsic sterilized polypropylene hub 19 to encase the sterile cannula. The most general mechanism, being the body mechanisms, consists of the general support and powers of the device, which may include but is not limited to body support beams 6, a ph skin color configuring adhesive 7, a battery replacement and containment point 8, a printed circuit board 9, and a PCB center data chip 10. The process of administration using this device begins with the application of the device to the epidermis of the skin using the pH skin color configuring adhesive 7. The adhesive, made of a skin-like silicone material, utilizes a pH-activated dye that reacts with the acidity in the users’ skin and configures the color of the adhesive to best match the user’s skin color, establishing an inclusive and natural appearance of the adhesive on the skin. The adhesive exists as a two-step application in which the user may apply the silicone adhesive to the device and then attach the device to the upper layer of the skin on an applicable absorption site.

Once the adhesive is thoroughly applied, a 38 mm replaceable cannula 11 may be inserted via the cannula replacement port 15. The port 15 utilizes an outer-body rubber flap insertion, with the workings of 6-connecting triangular flaps to allow the sterile cannula injector 18 to safely deliver a sterile cannula to the device. When the injector 18 is inserted into the holdings of the port-flaps 15, the flaps push slightly inwards into the gap between the drug cartridge 14 and device encasing 1 and allow the injector 18 to place the sterile cannula 11 into the device. Once the injector 18 is removed, the flaps 15 return to their original position to provide an additional blockage and gap-area between the cartridge 14 and cartridge expander gates 13 and the outside body. The injector 18 houses an intrinsic sterilized polypropylene hub 19 to provide a thin outer covering to the replacement cannula 11 during the risk of exposure to the subcutaneous cannula 11 during insertion.

Besides the port mechanisms, cannula insertion works along with several internal factors. Example to the procedure begins with a device command on the mobile app, permitting the opening of the bottom and top cartridge expander gates 13 to open outwards across the outer cartridge 14 edge. This opening is caused by the wireless communication between the mobile app and the device’s printed circuit board 9 and center data chip 10 to command the left 17 and right 12 motors to contract towards each other, being inwards, to open the gates 13 or release away from each other, being outwards, to close the gates 13. The inwards contraction causes both cartridge gate expanding crossbars 16 to push the upper and lower cartridge gates 13 outwards, thus opening the port for cannula insertion. Similarly, the outwards release causes the crossbars 16 to push the upper and lower cartridge gates 13 inwards, thus closing the top port entirely and closing the bottom port around the cannula after cannula insertion. For insertion, the cannula may be placed through the cannula replacement port 15 and through the top and bottom cartridge gates 13, then allowing the gates 13 to immediately relax as the cannula is released into the holdings of the relaxed left 17 and right 12 motors. The user may select a command on the devices’ mobile app that may trigger the left 17 and right 12 motors to contract and remain contracted, thus forcefully thrusting the cannula 11 into the subcutaneous layer of the skin. Automated controls and distribution controlled by the printed circuit board 9 and center data chip 10 may periodically deliver prescription medication doses as programmed by a provider in the devices’ mobile app.

The next step in application would be to utilize the drug injection syringe 2 to insert a desired amount of liquid drug into the 10.1 mL/1010 IU drug cartridge 14 through the drug cartridge injection and refill point 3. The drug cartridge injection and refill point 3 utilizes a push door mechanism that works with an outer-body rubber-flap insertion, similar to the cannula replacement port 15, and a more secure inner body secondary port 20 to further contain the drug. The secondary port 20 consists of a hinged gate with expansionary hinge springs to prevent inward release of the gates and leakage of the drug. In addition, the left gate of the secondary port 20 includes a thin expansion bar to secure the gates together and close any persisting gaps, if any. During insertion of a drug from the drug injection syringe 2, the syringe 2 may push through the holdings of the rubber-flap insertion of the drug cartridge injection and refill point 3 and into the gates of the secondary port 20, thus pushing the gates open for entry of the drug. When sufficient drug has been administered to the 10.1 mL/1010 IU drug cartridge 14, the drug injection syringe 2 may be retracted from the port 20, thus relaxing the expansionary hinge springs and resealing the injection site 3 and securely containing the liquid drug within the 10.1 mL/1010 IU drug cartridge 14 until further cleaning or refilling.

Once the medication refilling in the drug cartridge 14 empties, an exemplary micro ultrasonic level drug cartridge content level sensing transmitter 4 leveled across the interior right side of the cartridge 14 may be used to detect content levels and trigger a notification to the devices’ mobile app for the user to clean and refill the cartridge. The sensor 4 uses connective wires that travel encased within a right body support beam 6 and connects to the printed circuit board 9 and center data chip 10 to communicate wirelessly with the user’s mobile app. Additionally, the battery replacement and containment point 8 conductively hosts 20×3.2 mm CR2032 button cell batteries that may power the entire device and respective mechanisms. The entire body of the drug cartridge 14 and upper mechanisms of the device are supported along the bottom of the device with three distributed body support beams 6. Additionally, the drug cartridge 14 is further supported along the top of the device with 2 bilateral cartridge support bevels 5.

The above description is given by way of example, and not limitation. Given the above disclosure, one skilled in the art could devise variations that are within the scope and spirit of the device disclosed herein. Further, the various features of the embodiments disclosed herein can be used alone, or in varying combinations with each other and are not intended to be limited to the specific combination described herein. Thus, the scope of the claims is not to be limited by the illustrated embodiments.

1. A device-encasing body.

2. The body of claim 1, wherein a drug injection syringe administered and provided by a separate provider may administer the prescribed drug.

3. The body of claim 1, wherein a 10.1 mL/1010 IU drug cartridge contains the liquid drug.

4. The drug cartridge of claim 3, wherein a drug cartridge injection and refill point utilizes a push-door mechanism to allow drug refillment to the device.

5. The drug cartridge of claim 3, wherein a micro-ultrasonic drug cartridge content-level sensing transmitter with an encased and wired PCB connection scheme may detect when the devices’ drug levels are running low and in need of refillment.

6. The drug cartridge of claim 3, wherein cartridge support bevels provide support and structural anchoring of the drug cartridge to the walls of the device.

7. The drug cartridge of claim 3, wherein exists a secondary port to drug insertion, consists of a hinged gate with expansionary hinge springs to prevent inward release of the gates and leakage of the drug in the drug cartridge.

8. The drug cartridge of claim 3, wherein a set of cartridge gate expansion crossbars, corresponding to the top and bottom cartridge gates, widens and closes the cartridge gates when needed.

9. The drug cartridge of claim 3, wherein cartridge expander gates located on the top and bottom of the drug cartridge work to open and close to grant cannula replacement.

10. The body of claim 1, wherein body support beams are structured to support the upper and lower sections of the device.

11. The body of claim 1, wherein a pH skin color configuring adhesive connection of which colors the adhesive of the device in accordance to the patient’s skin-PH and securely connects the pod to the patient’s skin.

12. The body of claim 1, wherein a battery replacement and containment point for accessible battery configuration and holding exists.

13. The body of claim 1, wherein a printed circuit board electronically connects the devices’ body operations together.

14. The PCB of claim 13, wherein a center data chip operates bluetooth, inter-device connection, and control functions of the device.

15. The body of claim 1, wherein a 38 mm replaceable cannula exists to transfer the drug intradermally.

16. The body of claim 1, wherein a right motor with panels connected to the printed circuit board to contract the right crossbars inwards and relax them outwards mechanizes for cartridge gate operation; via connection to a Bluetooth mobile app.

17. The body of claim 1, wherein a cannula replacement port, utilizing a push-door mechanism, accepts and releases replacement cannulas.

18. The body of claim 1, wherein a left motor with a connection band, center motor panel, and PCB attached push band push the left crossbars inwards.

19. A sterile cannula injector utilizing a pen-like mechanism insertion for injecting a sterile cannula replacement into the device.

20. The injector of claim 19, wherein an intrinsic sterilized polypropylene hub protects the cannula further upon insertion to the device body.
